(19) United States
(12) Patent Application Publication
(10) Pub. No.: US 2023/0404627 A1
(43) Pub. Date: Dec. 21, 2023

(54) A MODIFIED COMPRESSION APPLIANCE AND ASSOCIATED HARDWARE FOR USE IN SPINAL SURGERY

(71) Applicants: RETROSPINE PTY LTD, Sydney (AU); Kevin SEEX, Sydney (AU)

(72) Inventor: Kevin SEEX, Sydney (AU)

(21) Appl. No.: 18/249,653

(22) PCT Filed: Oct. 20, 2021
(86) PCT No.: PCT/IB2021/059664

§ 371 Date: Apr. 19, 2023

(30) Foreign Application Priority Data
Oct. 20, 2020 (AU) 2020903788

(51) Int. Cl. A61B 17/70

(52) U.S. Cl. CPC A61B 17/7034

(57) ABSTRACT

A compression assembly for positioning spinal vertebra to which are attached anchors on which is mounted a lockable primary rod movable relative to the anchors. The assembly comprises a compression tool capable of inducing a compressive force to said anchors to enable said positioning of said vertebra. The compression tool having first and second arms each having a distal working end which in use, a first of said arms engages the rod. Each said first and second distal working ends of the tool, each have a formation which co-operates with the rod; the formation on the second arm by co-operation with an anchorage associated with or on the primary rod enabling a concurrent rotation of the rod and translation of a vertebra to which the rod is connected via the vertebral anchors during compression applied to said anchors by the compression tool prior to locking of the rod by the vertebral anchors when vertebrae are in a selected position.

### BACKGROUND

The present invention relates to surgical aids, tools and appliances and more particularly, relates to a combined compression and rotation device which is used in particular though not exclusively in spinal surgery. The invention further relates to a device which enables compression of spinal vertebra while allowing translation of the vertebra. More particularly, the present invention relates to a surgical tool which improves the versatility and range of adjustment of bony anchors such as pedicle screws for setting vertebral bodies to correct spinal deformities. The present invention further provides a compression tool having formations on the end of distal arms which engage bony anchors in a way that allows movement of the anchors in multiple directions and holding the anchors at a selected position/orientation to facilitate more accurate setting of vertebral bodies of the spine. The invention further provides modified primary rods used in spinal fixation and which accommodate and co-operate with the modified formations on the compression tool. The invention also provides a kit comprising a tool according to the invention and an arrays of rods which include formations such as openings which also receive the formations on the tool.

### PRIOR ART

There are in existence a number of assemblies used for setting of vertebral discs during posterior spinal surgery to achieve optimal lordosis. A commonly performed spinal procedure is an intervertebral spinal fusion procedure that typically involves the steps of removing a portion or all of the affected disc material, spreading apart adjacent vertebrae with a distractor, and inserting an implant bone or cage or prosthetic disc into the space previously occupied by the removed disc material. This procedure can be performed on the lumbar spine from the back (posterior fusion). Posterior surgery employs relatively large tools since the insertion space is more accommodating and posterior structures requiring retraction i.e. muscles are less sensitive.

The prior art is replete with a variety of instruments for spinal surgery including combined compressors and distractors and instruments specifically for distraction and/or retraction in spinal surgery. In one example U.S. Pat. No. 6,017,342 discloses a compression and distraction instrument having two pivotally connected handles. Jaw portions engage objects, such as human bone, for purposes of manouvering.

In another example U.S. Pat. No. 6,712,825 discloses a spinal disc space distracter for separating adjacent elements, such as vertebrae. The distracter preferably has a scissors-type distracting mechanism, either in a simple scissors or double-acting scissors configuration. The distracter includes blades that are removable from the jaws of the distracter such that different blades may be used depending on the patient and situation with which the distracter is to be used. The jaws include a mating fixture and the blades include a mating portion for removable association with the mating fixture. The jaws and handles are pivotally coupled together in a double-acting scissor-like configuration to further reduce the space required to move the jaws apart and thus further minimize the invasiveness of the device and procedure.

Various prior art documents disclose assemblies for spinal fixation. For example, CA2837699A1 discloses a compression and distraction spinal fixation system and kit. There is disclosed a screw and rod system teaching the use of a ratchet system to enable a surgeon to maintain compression until the screws are tightened against the rod. This apparatus does not provide any facility for combined compression and rotation and disc translation or separate rotation enabled by fixation to the rod of an arm of the compressor.

US2002/0123754 discloses an instrument for bone distraction and compression which has been fitted with ratcheting tips that are rotatably mounted at the working end of a bone distractor. The ratcheting tips are mounted such that they are adjustable to customise the instrument for a particular surgical procedure. The tips may be used to push implants along the rod. The tips can include U shaped ends that slide around the rod, hooks or other components to urge the two to desired positions. There is no teaching in this disclosure of a capacity to use the instrument to engage bony fixation and to enable co incident compression and rotation (translation of vertebra) for a surgeon to obtain optimal lordosis.

US2010/0331849 discloses a surgical instrument for moving tissue such as bone segments of a spinal column and enabling spinal compression and/or distraction for axial repositioning of vertebrae. US2011/0106168 discloses a laminoplasty rod and rod system that allows for variable angulation, translation (described as distraction and/or compression) and rotation of a spinal lamina bone portion associated with a laminoplasty prior to fixation thereof. US2011/0116467 discloses tools useful in fitting a spinal stabilisation system in a patient. An instrument is disclosed for use circumstances where spinal vertebrae may require compression or distraction. A driver engages a screw which enables a surgeon to compress or distract one or more levels of the vertebrae in parallel motion. Although the known compression tools allow standard compression to achieve lordosis, they merely sit on a rod but have no means to provide a capacity to effect translation of vertebrae.

In addition to the aforesaid prior art there are currently devices and appliances for use in posterior spinal surgery which enable rotating compressors, distractors and rods to correct deformity. Tools known as compressor distractors are currently used in spinal surgery. Compression draws adjacent vertebra towards each other and distraction is an opposite action which draws the vertebra apart. The aim of spinal fusion surgery is to restore a patent's spine to normal lordotic position. This is difficult to achieve using current instruments. The techniques enabled by the current instruments frequently fail to restore enough lordosis resulting in faster degeneration of the next segment (known as adjacent segment disease) leading to more surgery.

Anterior posterior and lateral spinal fixation is commonly used for the treatment of degenerative disease trauma deformity of the spine. The current state of the art includes the placement of bone screws into the vertebral bodies of spinal segments. The bone screws are connected to each other by rigid longitudinal primary rods to stabilise the spine. The bone screws adopt polyaxial screw head technology which allows more complex rod placement and screw connection. The screw and rod system allows fixation of vertebral segments but achieving normal lordotic positioning can require distractive, compressive and translational adjustment of vertebra to achieve the normal position and therefore spinal balance.

The metallic rods are solid and cylindrical and connect to pedicle screws via openings in the screws which are anchored to vertebral bone. When the screws are inserted in bone, the rods are passed through the openings in the screws which are then tightened against the bone. Prior to tightening the surgeon must use the current instrumentation to compress the vertebra which the screws are anchored in to set a desired position. Ideally this involves combined compression and translational movement but the current instruments only allows compression but not translation. An ideal instrument is one that would compression and translation at the same time to enable fine adjustment of the respective vertebral positions, but there is no known instrument which can achieve this objective.

In normal spines the facets joint have an oblique curved orientation to their matching internal joint surfaces. The normal spine motion of extension translates one vertebrae on another by a few mm, which realigns these surfaces to allow greater extension. The surfaces of the joints have a similar role in spinal flexion. During the compression stage of spinal fixation (traditionally thought to be the point at which the surgeon creates lordosis), the motion of extension is simulated but traditional instrumentation and methods do not permit any vertebral posterior translation which, unless the facets joints have been removed, limits the amount of compression (and thereby lordosis) that can be achieved. By allowing simultaneous posterior translation of one vertebrae on another during compression the intact facets joints are kept “unlocked” for longer allowing a greater amount of extension and thereby lordosis.

Typically the surgeon applies a compressive force between adjacent screws to increase lordosis to improve sagittal balance. Prior to that, the surgeon may distract bone to gain access to the intervertebral disc spaces for removal of disc, and insertion of spacers and bone graft materials both for support and to aid fusion. In the past, using current instruments, one surgeon must apply compression forces while another surgeon or assistant tightens the screws in the position determined by the surgeon. As compression is applied, ideally a rotational force is also required to achieve optimal lordosis but the current instrumentation does not allow this advantage.

There are limitations inherent in the conventional compression/distraction instruments used widely in spinal surgery and routinely in posterior spinal surgery. Consequently, there is a long felt want to improve the instrumentation and techniques used to achieve optimal and normal lordotic position to improve long term outcomes from spinal surgery and to reduce post-operative complications.

Current tooling applies compression forces from endplates onto cage/graft in the disc space and thereby pivots vertebral body on the cage to gain lordosis across the disc space. This normally Requires one tulip/anchor to be locked at start to prevent rod rolling over and rod sliding out of the tulip. Locking of the tulip also locks the rod to a particular position.

The nature of engagement between the compressor arm and primary rod allows downward pressure onto the rod compression. Otherwise, the compressor will slide up and off the rod, which can be dangerous causing injury to anatomy. Two pairs of hand are required to concurrently control position, apply compression forces and lock the tulips. Ideally surgeons should compress both sides at the same time, but this is practically impossible with known instruments. Locking the tulip locks the rod, which limits tulip ability to slide, as it has to follow the rod. This compromises gaining of optimal lordosis. Prior art rods have had openings, but they are merely to secure the rod to its holder and have no role in co-operation with compressor arm. This hole is not used to allow the rod to rotate in same plane as a long plane of rod. There is a need to provide improvements to tooling which can enable combined compression and translation for obtaining optimal lordosis.

### INVENTION

With the aforesaid prior art problems and disadvantages of current methodologies in mind, the present invention provides improvement in devices used in spinal surgery which enable combined and concurrent compression of spinal vertebrae and translation. The present invention according to one embodiment, provides an assembly including a tool which enables a combined compression and sagittal plane rotation for setting spinal vertebrae which is used in particular though not exclusively in spinal surgery. The invention further provides a device which enables compression of spinal vertebrae while allowing translation of the vertebrae during locking of screws against rods in a screw and rod spinal fixation assembly. The present invention also provides surgical tool which improves the versatility and range of adjustment of bony anchors such as pedicle screws for setting vertebral bodies to correct spinal deformities.

The present invention further provides a tool having formations which engage bony anchors in a way that allows movement of the anchors in multiple directions and holding the anchors at a selected position/orientation to facilitate more accurate setting of vertebral bodies of the spine. The invention also provides an assembly comprising a tool having a distal end formation on at least one of its arms which engages a primary rod or an attachment to a primary rod and which enables concurrent compression and translation.

Outlined broadly below are embodiments and features of the invention to enable the invention to be better understood, and in order that the present contribution to and improvement over the current the art may be better appreciated. There are, of course, additional features of the invention that will be described hereinafter and which will form the subject matter of the claims.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways in various anatomical sites including in veterinary applications. Also, it is to be understood that the phraseology and terminology employed herein are for the purpose of description and should not be regarded as limiting. As such, those skilled in the art will appreciate that the conception, upon which this disclosure is based, may readily be utilized as a basis for the designing of other variations on the tool assembly, structures, methods and systems for carrying out the purposes of the present invention.

It is therefore an object of the present invention to provide a new and improved tool and associated hardware which removes the practical disadvantages encountered using current instrumentation and techniques. It is a further object of the invention to provide a modified compressor arm which works in conjunction with a primary fixation rod to allow a user to effect translation of a vertebra during compression by the compressor. It is a further object of the invention to provide a modification to the primary rod which enables an engagement with a modified compressor arm and which also enables combined compression and translation by the compressor.

In one broad form the present invention comprises: a compression tool for use with a spinal fixation system including screws and co-operating primary fixation rods, the tool comprising first and second handle portions each having a first end with a gripping formation and a second working end, the first and second handle portions pivotally coupled to each other via a pivot connection enabling the tool to operate in scissor like fashion, characterised in that the working end of each handle portion includes at least one formation capable of direct engagement with said rods or screws allowing the tool to transmit a compression and rotational force to the rods via the at least one formation; wherein the formations are arranged to allow compression and translation of spinal vertebrae using the tool.

According to a preferred embodiment the formations allow the rotation when the tool is rotated in a plane through which the scissor action operates. According to one embodiment, the formation comprises a post which laterally extend from the working ends. Preferably the post is capable of movement about a transverse axis through the post, the rotation induced when the tool is applying compression and rotation to the primary rods of the spinal fixation system. According to an alternative embodiment, the working formation includes an opening, such as a circular formation which engages around the primary rod. In a further embodiment the working formation comprises a hook which engages around the primary rod. According to a preferred embodiment the assembly works in conjunction with at least one implantable primary rod and which is connected to pedicle screws.

The rod may be modified with a formation such as but not limited to an opening which engages with a formation on the compressor. According to one embodiment. the opening in the primary rod is at one end of the rod, preferably at the caudal end. In another embodiment the opening is at or near the mid length of the rod. The assembly further comprises a compressor having fixed or detachable working modular arms with working ends including a rod formation (fixed or rotatable) which engages securely into the at least one opening/hole in the primary rod. This provides a reasonably stable and secure fulcrum for rotation of a compressor and it prevents rod roll over and the rod formation sliding out of compressor during use. The remainder of the primary rod lies longitudinally within screws, although screws remain unlocked during compression unlike in the prior art where the rod is locked with one screw prior to compression.

The opposite end of the modular compressor includes a horizontal hook that fits under the primary rod and engages the pedicle screw. In this embodiment, the hook engages underneath the primary rod and enables lifting of the rod (sagittal plane rotation), while the compressor applies compression forces to the screws. The major advantage is that this hook can be used not only at the end of a construct but also between screws. In an alternative embodiment, the aforesaid arrangement is adapted to a similarly designed distractor but including the hook facing cranially. According to one embodiment the rod formations engage an opening in the screw. According to an alternative embodiment, the rod formations engage an opening in the rods of the fixation system.

In another broad form the present invention comprises: a compression and distraction tool for use with a spinal fixation system including screws and co-operating rods, the tool comprising first and second handle portions each having a first end with a gripping formation and a second working end, the first and second handle portions pivotally coupled to each other via a pivot connection enabling the tool to operate in scissor like fashion, characterised in that the working end of each handle portion includes a transverse post which is capable of rotation relative to a distal end of the handle portion; thereby allowing the tool to rotate about an axis through the post formation.

In another form the present invention comprises; a spinal rod for use with a rod and screw assembly for setting the position of spinal vertebra under the influence of a compression tool, the compression tool including an arm having a working formation which fixedly engages the rod; the rod comprising at least one formation thereon which co operates with the working formation on the compressor arm thereby enabling concurrent compression and translation of spinal vertebrae.

In another broad form the present invention comprises: a detachable arm for a compressor tool for use with a spinal fixation assembly including, bone anchor screws and at least one co-operating primary rod, the compressor having scissor action first and second handle portions each having a first end with a gripping formation and a second working end, the first and second handle portions pivotally coupled to each other via a pivot connection enabling the tool to undergo the scissor action, characterised in that the working end of each handle portion of the tool includes a connection abutment each of which detachably retains a proximal end of a working arm; at least one said working arm including a distal formation which engages the co-operating primary rod which resists separation between the rod and formation when the compressor is rotated to pull up on the rod thereby enabling combined compression and translation of vertebrae.

According to a preferred embodiment, the assembly includes a plurality or kit of working arms each having its distal end formation configured for different forms of engagement with the primary rod. Distal end formations which engage the primary rod include a hook, a slot such as but not limited to a U shaped slot, an opening which receives the primary rod in male female inter fitting, a strut or rod extending preferably normal to a longitudinal axis of the working arm.

The formation is capable of engagement with said screws allowing the tool if a compressor is selected to transmit a compression force to the screws via the formation; wherein the formations are arranged to allow relative movement between the handle portions and the formations when a rotational force is applied to the compression tool. In another embodiment, the formation is capable of engagement with said screws allowing the tool if a distractor is selected to transmit a distraction force to the screws via the formation; wherein the formations are arranged to allow relative movement between the handle portions and the formations when a rotational force is applied to the distraction tool.

In another broad form the present invention comprises:
a compression assembly for positioning spinal vertebra to which are attached anchors on which is mounted a lockable primary rod; the assembly comprising a compression tool capable of inducing a compressive force to enable said positioning of said vertebra; the compression tool having first and second arms each having a distal working end which in use, engages the rod; characterised in at least one said first and second distal working ends of the tool, have a formation which co operates with the rod; at least one said formations by its engagement with the rod enabling a translation of a vertebra during compression applied to said anchors to enable compression of vertebrae prior to locking of the rod by the anchors against relative movement.

In another broad form, the present invention comprises:
an attachment for detachable attachment to a primary rod used in spinal fixation and which is retained by pedicle screws; the attachment co operating with a compression tool which in use positions spinal vertebra by acting on anchors attached to vertebra; the compression tool capable of inducing a compressive force to said anchors to enable said positioning of said vertebra; the compression tool having first and second arms each having a distal working end; wherein one said distal end engages the attachment to the rod via a formation on the arm; the engagement via the formation on the arm and attachment to the primary rod enabling a rotation of the rod and translation of a vertebrae during compression applied to said anchors prior to locking of the rod by the anchors.

According to a preferred embodiment the attachment comprises a body having jaws defining a recess which receives and engages a primary rod when mounted on pedicle screws. The attachment also includes a formation which accommodates a formation on a distal end of a compressor arm. The attachment is lockable against the rod by such means as a grub screw or the like.

Preferably the anchors comprising pedicle screws. The primary rod according to one embodiment is a standard rod with no modification and which receives the detachable attachment. In that case the compressor arm formation engages a formation on the attachment to facilitate the ability to pull up on the rod and effect vertebral translation. According to another embodiment, the rod includes an integral formation preferably at one end which receives the formation on the compressor arm. Formations contemplated on the primary rod to accommodate the formation on the end of the compressor am are: an opening, a groove, a slot, an abbreviation, a recess, a through hole, indent.

These together with other objects of the invention, along with the various features of novelty which characterize the invention, are pointed out with particularity in the claims annexed to and forming a part of this disclosure. For a better understanding of the invention, its operating advantages and the specific objects attained by its uses, reference should be had to the accompanying drawings and descriptive matter in which there is illustrated preferred embodiments of the invention.

The present invention provides an alternative to the known prior art and the shortcomings identified. The foregoing and other objects and advantages will appear from the description to follow. In the description reference is made to the accompanying representations, which forms a part hereof, and in which is shown by way of illustration specific embodiments in which the invention may be practiced. These embodiments will be described in sufficient detail to enable those skilled in the art to practice the invention, and it is to be understood that other embodiments may be utilized and that structural changes may be made without departing from the scope of the invention. In the accompanying illustrations, like reference characters designate the same or similar parts throughout the several views. The following detailed description is, therefore, not to be taken in a limiting sense, and the scope of the present invention is best defined by the appended claims.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 shows a perspective view of a compressor tool according to one embodiment.

FIG. 2 shows a side elevation view of the compressor/distractor of FIG. 1.

FIG. 3 shows a side elevation view of a screw and rod assembly anchored to vertebrae, showing the rod having an opening to receive a distal formation on the compressor/distractor arm

FIG. 4 shows with corresponding numbering an opposite side view of the screw and rod assembly of FIG. 3;

FIG. 5a shows a perspective view of a primary rod according to one embodiment and including an end opening which in use engages a formation on the compression and distraction device;

FIG. 5b shows a top view of the primary rod of FIG. 5a;

FIG. 5c shows a side view of the primary rod of FIG. 5a;

FIG. 5d shows an underside view of the primary rod of FIG. 5a;

FIG. 5e shows an end view of the primary rod of FIG. 5a.

FIG. 6 shows a set of primary rods according to another embodiment and each including an end opening and which in use engages a compression arm;

FIG. 7 shows a perspective view of a compressor device according to an alternative embodiment.

FIG. 8 shows an elevation view of the compressor of FIG. 7.

FIG. 9 shows the perspective view of the compressor of FIG. 8 with working arms attached to respective abutments.

FIG. 10a&b shows a selection of working arms with different distal end configurations.

FIG. 11 shows a side elevation of a rod and screw assembly with scissor compressor and connected to its connecting abutments, working arms.

FIG. 12 shows the assembly of FIG. 11 having undergone compression adjustment responsive to scissor action of the compressor.

FIG. 13 shows a side elevation of a rod and screw assembly including scissor compressor and connected to its connecting abutments working arms with their positions reversed relative (compared to the arrangement shown in FIGS. 11 & 12) to the anchor screws.

FIG. 14 shows a side elevation of a rod and screw assembly including scissor compressor and connected to its connecting abutments working arms alternative working arms.

FIG. 15 shows a perspective view of a compressor arm with an alternative formation for engagement to a primary rod.

FIG. 16 shows an enlarged exploded view of the connection arrangement in FIG. 15.

FIG. 17 shows an assembled view of the alternative compressor arm.

FIG. 18 shows an enlarged assembled view of distal end of arm and bridge and slot.

FIG. 19 shows a perspective exploded view of an alternative connection arrangement between a distal end formation of a compressor arm and the primary rod.

FIG. 20 shows with corresponding numbering an enlarged exploded view of the attachment prior to engagement with a primary rod.

FIG. 21 shows an opposite side view of the exploded view of FIG. 20.

FIG. 22 shows the attachment locked to the rod with post of compressor arm separated from the attachment.

FIG. 23 shows an enlarged end view of the attachment locked to primary rod and post formation engaged with the attachment.

### DETAILED DESCRIPTION

The present invention will now be described in more detail according to a preferred embodiment but non limiting embodiment and with reference to the accompanying illustrations. The examples referred to herein are illustrative and are not to be regarded as limiting the scope of the invention. While various embodiments of the invention have been described herein, it will be appreciated that these are capable of modification, and therefore the disclosures herein are not to be construed as limiting of the precise details set forth, but to avail such changes and alterations as fall within the purview of the description. Although the invention in one form will be described with reference to transverse rods extending from the compressor it will be appreciated by persons skilled in the art that alternative formations which achieve the inventive objects are contemplated, including the shape, angle, size orientation and material of construction of the compressor formations. A reference to a primary rod will be a reference to a rod used in co-operation with a pedicle screw or the like in a spinal rod and screw assembly.

Restoration of the correct shape of the spine is one of the modern metrics of a successful lumbar spinal fusion operation. However despite this knowledge, numerous studies show that fusing the spine in a suitably lordotic shape is often not achieved. Failure to fuse the lumbar spine with adequate lordosis can result in increased pain, worse quality of life and more surgery for adjacent segment disease. Currently, achievement of optimal lordosis is to a significant extent influenced by limitations on the compressor instrument characteristics and features and vertebral motion in fusion. The present invention to be described in detail below, provides a compressor tool having working ends which optimise vertebral motion in fusion to thereby optimise lordosis. This is enabled by formations on the working end or ends of the compressor which engage the rod so that the tool can apply a rotational force to the rod at the same time as application of a compression force.

The engaging formation enables an engagement with the rod which in turn enables optimisation of motion segment behaviour during the compression phase of spinal fusion. This enables an increase in the extent of lordosis. According to one embodiment, the rods may be adjusted to accommodate the formations on the distal working ends of the arms of the compressor. According to a preferred embodiment the modified compressor works in conjunction with existing or modified rods—i.e. all tulip based pedicle screw systems with appropriate sized rods. One advantage of the present invention is that it enables additional rod movement control without having to lock one of the pedicle screws against the rod to prohibit the tendency for unwanted axial rotation of the rod during the compression step. More specifically it allows the new compressor to control the rod position without locking either tulip.

The modification to the compression tool described herein provides a mechanical advantage when compression is applied to pedicle screws prior to locking of a rod by the screws when a required optimal degree of vertebral lordosis has been achieved. The formation on the distal end of an arm of the compression tool resists separation between the arm and rod. The tool allows the surgeon to apply the required compression and at the same time sagittal rotation of the rod inducing a translation of one vertebra. The sagittal rotation of the rod during compression of the pedicle screws prevents unwanted axial rotation of the rod and unwanted displacement of the rod relative to the screws during positioning of the vertebra for the required lordosis.

According to the conventional technique, when compression of the screws is induced by a compression tool for setting the required lordosis (spinal curvature) the rod must be locked against one screw to avoid unwanted axial rotation/displacement and/or relative movement between the rod and screws. Using the modified tool according to the present invention there is no need to pre lock the rod to one of the screws as the sagittal rotation of the rod which induces translation of a disc and enabled by the formation on the distal end of one arm of the compression tool avoids relative movement between the rod and screws during setting of the required lordosis. A frictional resistance between rod and screw is achieved when the rod is rotated in the sagittal plane. This means that one surgeon can at the same time, apply the required compression and attend to locking of the screws without concern that unwanted displacement of the rod will occur during that process.

The present invention enables achievement of more lordosis that can otherwise be achieved using conventional compressors. The surgeon can achieve this using one hand which at the same time compresses and rotates (induced by pulling up on the rod) leaving the other hand free to tighten/lock the pedicle screws against the rod. Alternatively, the surgeon can simultaneously perform the aforesaid function of compression and rotation on two compressors at the same time which may require an assistant to tighten the pedicle screws to effect rod locking.

Although the invention is described herein with reference to various embodiments, a preferred embodiment provides an assembly which employs an implantable longitudinal rod which has an opening such as a recess, hole, slot, abbreviation, indent, at or near one end of the rod and a compressor having two arms one having a distal formation for pressing the rod down and the other arm having a formation which maintains engagement between the rod and arm formation when pulling up or pushing down on the rod.

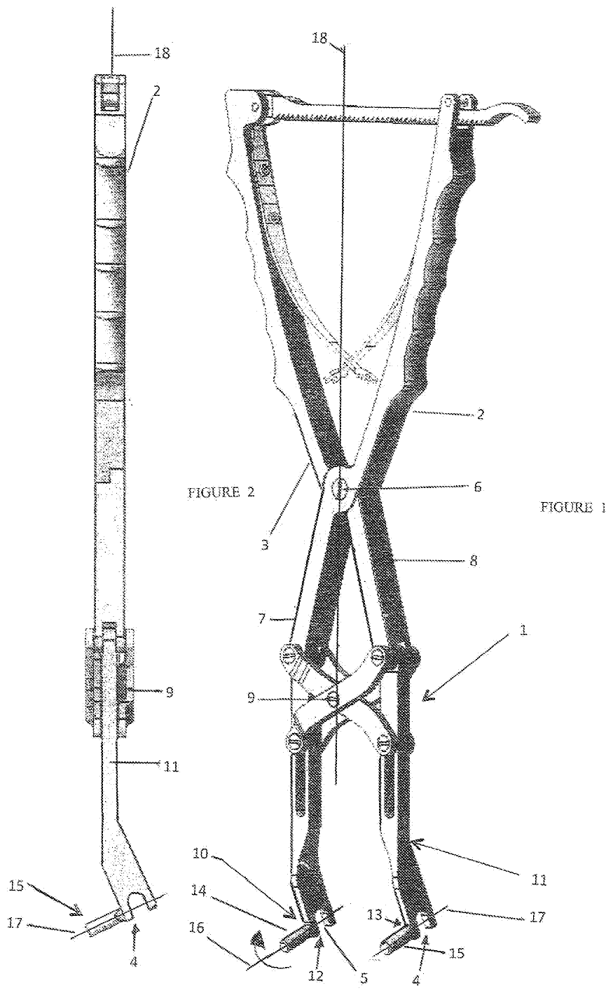

FIG. 1 shows a perspective view of a compressor device 1 according to a preferred embodiment. Device 1 comprises a scissor type body with a pair of handles 2, 3 pivotally connected together. Each handle 2, 3 respectively terminates at a distal end in opposing jaws 4 and 5 and in use squeeze the jaws together. The jaws 4 and 5 are pivotally coupled together in a double-acting scissor-like configuration. Handles 2 and 3 pivot about first pivot 6 which has the effect of urging arms 7 and 8 towards each other. Second pivot 9 allows distal arms 10 and 11 to operate in parallel. In an alternative embodiment, scissor device 1 may be provided with only one pivot. The distracter preferably has a scissors-type distracting action, either in a simple single pivot scissor action or double-acting scissors configuration. Distal arm 10 terminates in a free end formation 12 and distal arm 11 terminates in free end formation 13. Formations 12 and 13 are characterised in that they include transverse rods 14 and 15. Post 14 according to one embodiment is pivotally attached to distal arm 10 and is capable of independent rotation relative to a plane through handles 2 and 3. Likewise post 15 according to an alternative embodiment is pivotally attached to distal arm 11 and is capable of independent rotation relative to the plane through which handles 2 and 3 operate handle 3. According to this embodiment, posts 14 and 15 rotate about respective transverse axes 16 and 17 relative to a longitudinal axis 18 through device 1.

In an alternative embodiment, posts 14 and 15 are fixed to distal arms 10 and 11 respectively but do not undergo rotation relative to arms 10 and 11. In that case their movement is in unison with respective distal arms 10 and 11.

According to one embodiment arms 10 and 11 may detachably receive and retain respective transverse posts 14 and 15. In this regard, the device can be fitted with the transverse rods as attachments allowing interchangeability of working ends. Alternatively arms 10 and 11 are detachable and include rods or other analogous formation as a working end to be used as described. The geometry of posts 14 and 15 allows their use in effecting distraction, compression and rotation about an axis parallel to the axes through the rods.

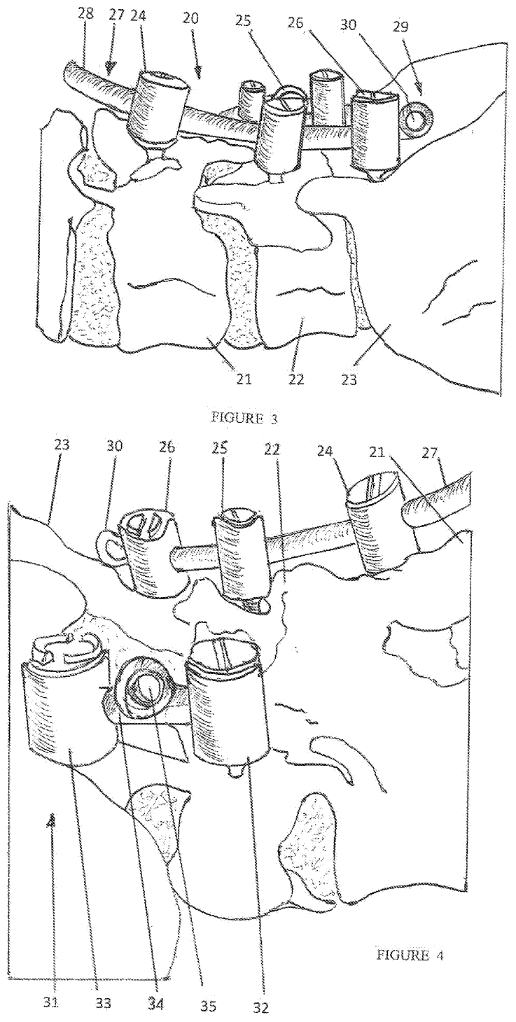

FIG. 2 shows with corresponding numbering a side elevation view of the Compressor of FIG. 1 but which can also act as a distractor. FIG. 3 shows a side elevation of a rod and screw system 20 attached to spinal vertebra 21, 22 and 23. System includes axially aligned screws 24, 25 and 26 respectively anchored to vertebral bone 21, 22 and 23. Passing through each screw 24, 25 and 26 is a rod 27 having free end 28 and opposite free end 29 which terminates in opening 30. The rod and screw arrangement shown is that typically used in spinal fusion surgery. Opening 30 accommodates posts 14 or 15 of compressor 1 and additional compression assemblies to be described below.

FIG. 4 shows an oppose side view of spinal vertebra 21, 22 and 23 including rod and screw assembly 20 showing rod 27 including opening 30 to receive a compressor distractor. In an alternative arrangement a parallel rod and screw assembly 31 is shown anchored via pedicle screws 32 and 33 into respective vertebra 22 and 23 retained by pedicle screws 32 and 33 is a primary rod 34 which includes an intermediate opening 35 which receives and retains rods 14 or 15 of CD 1. When CD 1 engages either of openings 30 or 35 via either one of posts 14 or 15 further engagement of the remaining rod with a pedicle screw will allow a surgeon to generate compression distraction rotation or a combination of those forces to manipulate the vertebra into a desired setting.

The present invention teaches an improved compressor and distractor device which includes formations to accommodate an opening in either a rod or pedicle screw to thereby enable more versatile manipulation of the spinal vertebra. Specifically, the transverse posts 14 and 15 are capable of engagement with opening 30 to allow both compression and rotation of vertebra at the same time. This enables a surgeon to more accurately position the screw and rod system 20 to achieve the correct lordosis. This arrangement allows the compressor distractor device 1 to connect with bony anchors such as screws 24, 25, or 26 as selected by a surgeon and with opening 30 in rod 27 to effect movement of spinal vertebra in multiple directions. For example, with one of posts 14 or inserted into a pedicle screw and the remaining other post (14 or 15 inserted into opening 30 a surgeon can compress compression of the pedicle screw and at the same time by virtue of the engagement of one of posts 14 or 15 with opening 30 to effect rotation of the spinal vertebra as device 1 moves through a plane transverse to the axis of posts 14 and Rod 27 after correct setting of the vertebra may be left in situ or removed. As the screws which are anchored in bone this causes the bone to move relative to the rod 27. A contemporaneous rotation of the rod 27 allows a close simulation of the anatomical translational behaviour and more accurate setting of the vertebra in their desired position. behaviour and practice device 1 can induce compression.

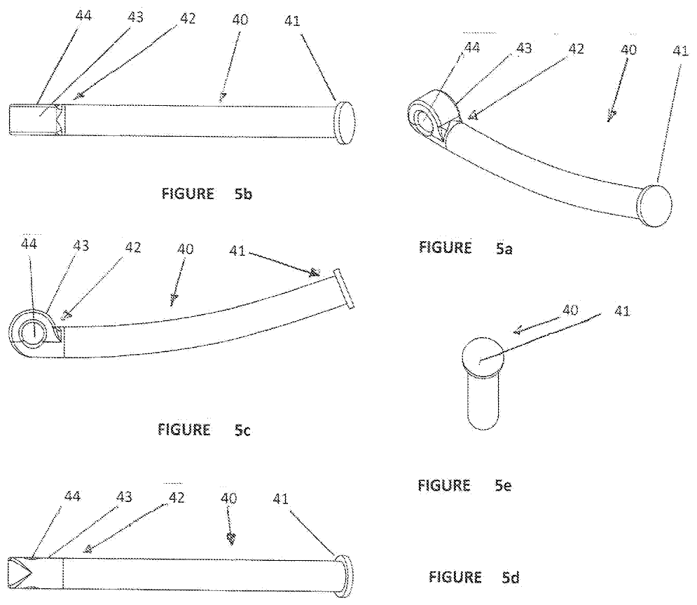

FIG. 5a shows a perspective view of an isolated primary rod 40 used in conjunction with a rod and screw fixation system as previously described. Rod 40 corresponds to the primary rod type 27 as described in FIG. 3. According to the embodiment shown rod 40 has a first end 41 and second end 42. At second end 42 there is provided a formation 43 which includes an opening 44 which in use engages a corresponding formation on a compression and distraction device of the type described in FIG. 1 or of the types to be described below. Specifically transverse posts 14 and 15 engage opening 44 so that when compressor device 1 is rotated in its own plane, compression and translation can be induced concurrently. FIG. 5b shows with corresponding numbering a top view of the primary rod of FIG. 5a. FIG. 5c shows with corresponding numbering a side view of the primary rod of FIG. 5a. FIG. 5d shows with corresponding numbering an underside view of the primary rod of FIG. 5a. FIG. 5e shows an end view of the primary rod of FIG. 5a. Rods may be modified from known rods as described in FIG. 5 or known rods can be used which receive and retain a detachable attachment to be described further below.

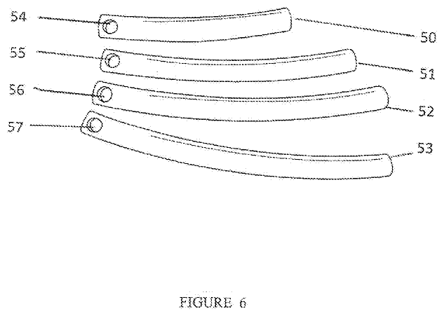

FIG. 6 shows an array of rods of varying lengths which are used in conjunction with a rod and screw fixation system as previously described in FIG. 4 corresponding to primary rod 34. Primary rods 50, 51, 52 and 53 have similar configurations with different lengths and radii. Rods are selected according to the requirements of the particular surgery. The selection may relate to the extent of lordosis required and the number of vertebrae being fused. Rods 50, 51, 52 and 53 respectively include openings 54, 55, 56 and 57. These openings, when the rods are in use, receive a formation on a compression tool arm which enables the tool to rotate the rod in a sagittal plane inducing translation of a vertebra to which the rod is indirectly attached, during compression applied for optimising lordosis. These rods may be manufactured from a material which allows a surgeon to apply bending to alter the primary rod geometry. Although the embodiments of the primary rods shown have one opening it is contemplated that rods can be provided with multiple openings indifferent positions along the rod and in a variety of lengths and radii and manufactured from various materials. In a further embodiment, the primary rod includes temporary or permanent sliders which contribute to the engagement of the compressor tool prior to application of forces to adjust the spinal vertebra. The primary rods may also be arranged to avoid neighbouring screws when in use. The rods may also receive a detachable attachment which receives a co operating formation on the compressor tool.

Compressor 1 can act parallel or at an angle to the long axis. posts 14 and 15 of device 1 can be adapted with a variety of geometries including angulated hollow tube, allen key engagement, alternative male female interfitting. A variety of dimensions are contemplated for the posts and the nature of the male female engagement can be interchanged. For example the primary rods may include a formation which engages and is retained by the compression device 1. It is contemplated that the co-operation between the formation/s on the primary rod and the formation/s of the compression device 1 is a connection allowing shear force transmission for strength. Device 1 according to one embodiment includes a ratcheting lock which enables setting of the device once engaged. The transverse post member connection to the primary rod allows relative rotation to occur between the formation of the primary rod and the co-operating engaging compressor post formations. As a result of this engagement the compressor is able to tilt and apply anteropostero forces in addition to Cranio caudal forces. Combined left and right instruments connected to screws can impart axial rotation forces to the spine. According to one embodiment, posts 14 and 15 are fixed to the compressor 1 without any relative rotation between the compressor and engaging formation extending therefrom.

In practice the device and assembly of the present invention in all its forms can be used according to the following methodology which is described by reference to a non limiting first example. One rod is attached to two bone anchors (pedicles) on one side of an exposed spine. A second rod is attached on the opposite side; for example rods are attached to poly axial tulips of screws entering pedicles of L5 and Si. Pedicle tulips are rotated to 90 degrees from a permanent position. Openings for rods lie transversely. The poly axial nature of most tulips allow engagement of the tool rods even if screws are not perfectly aligned. Different length rods allow engagement with tulips even if not in the same sagittal plane. Formations on the distal ends of compressor Device 1 (described previously as posts 14 and 15), fit into the tulip. Posts 14 and 15 can be locked to tulip screws by caps. According to one embodiment, the rod may rotate relative to the compressor due to rotational engagement with compressor arms (10 and 11). Each rod can be ‘locked’ to prevent unwanted withdrawal and to immobilise the tulip. Locking of tulips securely, prevents unwanted rod withdrawal and prevents unwanted head motion during compression. Alternatively, the rod can be left loose in the tulip and restrained from axial rotation by interaction with the compressor.

Another practical example of the operation of the assembly of the invention is described below. After attachment of one arm of the compressor device 1 to one selected anchor and the other arm to an adjacent anchor, a surgeon can pivot compressor 1 and pull up one anchor and at the same time push down an adjacent anchor. This can impart strong reduction forces in the sagittal plane. The surgeon can move and manipulate both sides of the spinal vertebra concurrently to achieve a variety of movements hereinbefore described. For example, if the left hand compressor is used to induce compression and a right hand side compressor is used to distract, this will correct coronal imbalance. On the other hand, if the left hand pushes superior vertebrae downwards while the right hand compressor pulls the superior vertebrae upwards, this will rotate superior vertebrae leading to correction of axial rotation. From the aforesaid, it can be seen that the improvements to the compressor device 1 described herein allow correction of complex deformity at one segment. Once the corrected position for the spinal vertebrae has been set, the spine can be held in position while temporary or permanent primary rods are inserted. This may be done one side of the sagittal plane at a time holding the spine in position with one of the compressor devices while adding a temporary or permanent rod to the contralateral side. The compressor is replaced with a permanent rod joining pedicle screws that is locked into position.

Various embodiments for the primary rods are contemplated to accommodate the formation on the distal end of a compressor device. For example, the primary rods may include multiple openings, alternative formations to the openings such as but not limited to, altering the male female interfitting, employment of alternative key in lock arrangements which may include rod widening, narrowing alternation of the cross section at the point of engagement with the compressor device, a hinged attachment or slide.

In a further embodiment, sliders may be attached to the primary rods connecting pedicle screws for manipulation. The slider location may be local or adjacent. A top opening in a slider receives and retains the device 1. A bottom opening receives and retains a longitudinal primary rod. A formation such as a but not limited to a transverse post on compressor 1 is inserted and the compressor is manipulated to move screws to a desired position at which point tulips are locked to a primary longitudinal rod to fix the spinal vertebrae in a desired position.

According to one embodiment, the sliders include locking fasteners which may for example be grub screws which allow relative locking between the primary rod and slider. With one compressor on each side of the spine, the compressor can be rotated, lifted up and pushed down. The use of the slider with the primary rods modified to accommodate the compressor allows the application of rotational, and compression forces alone or in combination.

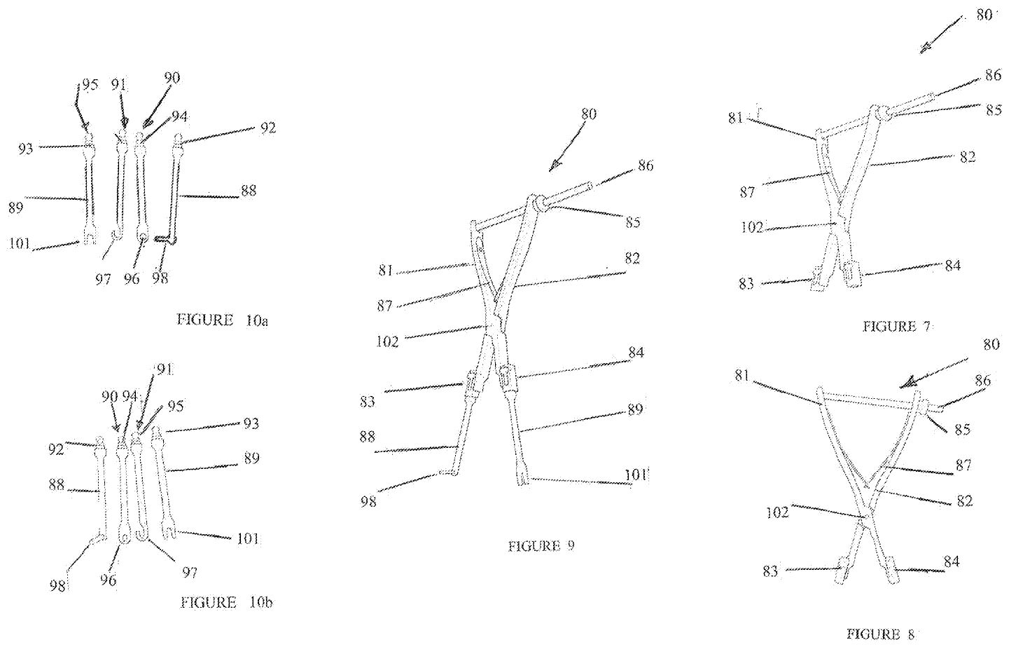

FIG. 7 shows a perspective view of a compressor device 80 according to an alternative embodiment. Device 80 comprises a scissor type body with a pair of handles 81 and 82 pivotally connected together by one pivot connection 102. Each handle 81, 82 respectively terminates at a distal end in connection abutments 83 and 84. Nut 85 mounted on threaded rod 86 allows scissor action opening and closing of opposing connection abutments 83 and 84. FIG. 8 shows with corresponding numbering, an elevation view of the compressor 80. Engaging handles 81 and 82 is spring 87 which biases compressor 80 to an open state in which abutments 83 and 84 are at maximum distance apart. Closing abutments 83 and 84 is effected against the action of the bias spring 87.

FIG. 9 shows the perspective view of the compressor 80 of FIG. 7 with working arms 88 and 89 attached to respective abutments 83 and 84. Working arms 88 and 89 are connected to the abutments via a threaded connection. It will be appreciated that other suitable forms of connection between the working arms 88 and 89 and respective abutments 83 and 84 are contemplated, such as but not limited to snap fit, slot and rod engagement etc.

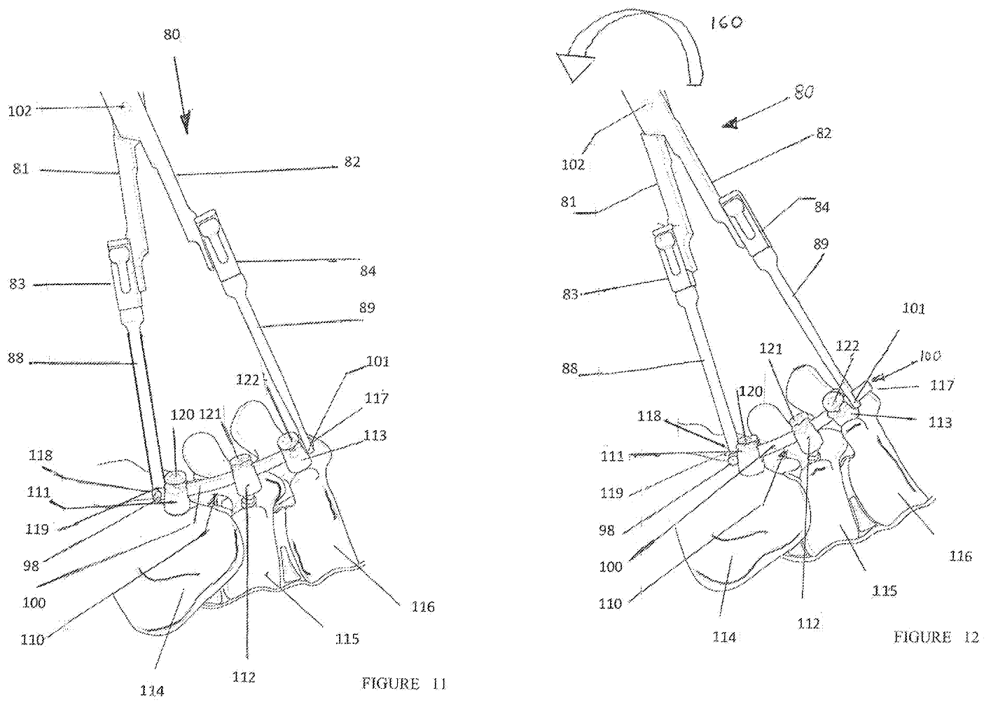

FIG. 10a&b shows a selection of working arms including arms 88 and 89 and arms 90 and 91. Working arm 88 has at its proximal end a threaded region 92 to enable screw in connection to an abutment. Likewise working arms 89, 90 and 91 have respective threads 93, 94 and 95. Working arm 88 has at its distal end a circular rod 98 which in use engages an opening in a primary rod 100 (see FIG. 13). Arm 89 includes at its distal end a U shaped formation 101 which engages primary rod 100. Similarly arms 90 and 91 include respective formations 96 and 97 configured to engage primary rod 100. Formation 96 allows working arm 90 to engage rod 100 via one end. Formation 97 is a hook which engages the rod from underneath. Arms 89, 90 and 91 are capable of sliding along rod 100. Arm 88 once engaged with rod 100 remains in that fixed relationship due to the engagement between rod 98 and primary rod 100. Rod 98 may be arranged to rotate during use, relative to shaft 99 of working arm 88. Rod formation 98 also enables compressor 80 when rotated to axially rotate rod 100. It will be appreciated that alternative distal formations, beyond those shown, are contemplated for the working arms.

FIG. 11 shows a side elevation of a rod and screw assembly 110 comprising scissor compressor 80 and connected to its connecting abutments 83 and 84, working arms 88 and 89. Arms 88 and 89 are connected to primary rod 100. Assembly 110 includes anchor screws 111, 112 and 113 respectively anchored to vertebral bone elements 114, 115 and 116. Passing through each anchor screw 111, 112 and 113 is rod 100 having a first end 117 and second opposite free end 118. Free end 118 includes an opening 119 which receives post 98 of arm 88. which terminates in opening 30. Opening 30 for instance could accommodates posts 14 or 15 of CD device 1. Working arm 89 is shown engaging first end 117 via formation 101.

Each of anchor screws include respective locking nuts 120 121 and 122 which allow each of screws 111, 112 and 113 to be locked to primary rod 100 or to be free to rotate relative to rod 100. This provides wide versatility to manipulate compressor 80 rotationally and along a scissor plane. Since each anchor screws 111, 112 and 113 are fastened to separate bone elements 114, 115 and 116 (usually vertebra), each can be selectively locked to rod 100 to prevent axial and rotational movement relative to rod 100. Locking of the anchors against rod 100 may be effected by using an Allen key. In the arrangement of FIG. 13 formation 101 of working arm 89 engages end 117 of rod 100. This places formation 101 adjacent anchor 113. Post 98 of working arm 88 is adjacent screw anchor 111.

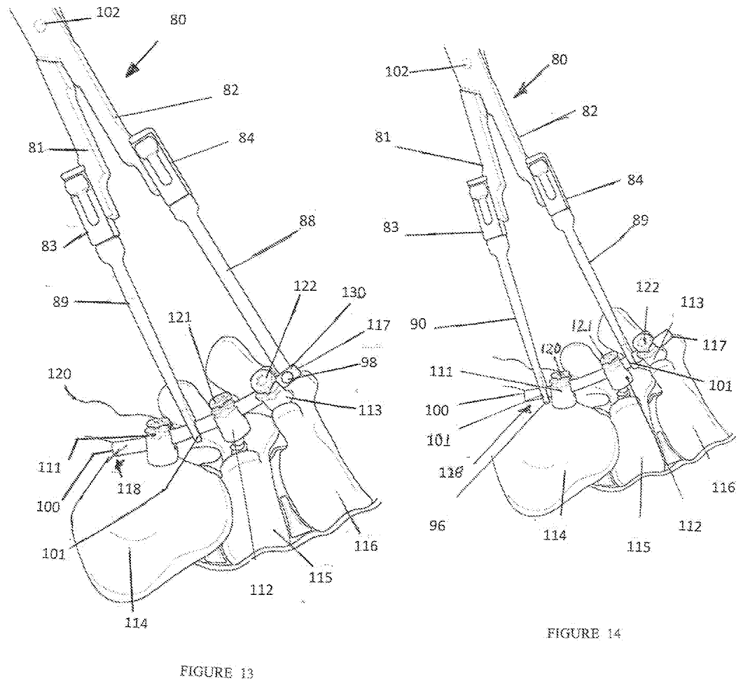

FIG. 12 shows the assembly 80 of FIG. 13 having undergone adjustment responsive to scissor action of compressor 80. Compared to the arrangement in FIG. 13, in FIG. 14, arm 89 has moved axially in the direction of arm 88. This has urged formation 101 axially along shaft 100 and at the same time has moved anchor 113 axially in the same direction taking vertebra 116 with it. This has narrowed the disc space between vertebra 116 and 115. At the same time, since anchor 112 is free relative to rod 100, vertebra 115, can move relative to rod 100. Under the scissor action of compressor 80 vertebra 114 and 116 are urged closer to vertebra 115.

This is one example of the numerous vertebral movement combinations which can be achieved using the assembly including lordotic adjustment by rotation of compressor 80 and selective locking of one or more of anchors 111, 112 and 113 against rod 100. FIGS. 13 and 14 show pre and post compression and the relative positions of anchors 111, 112 and 113. During compression, as indicated by arrow 120, for anchors locked to rod 100, there is a tilt of the spine, rod 100 and compressor 80 in the direction of arrow 120. After compression, end 117 is shown protruding beyond anchor 113.

FIG. 13 shows a side elevation of a rod and screw assembly 110 comprising scissor compressor 80 and connected to its connecting abutments 83 and 84, working arms 88 and 89 with their positions reversed relative (compared to the arrangement shown in FIGS. 11 & 12) to the anchor screws 111, 112 and 113 positions. Arms 88 and 89 are as before, connected to primary rod 100. Assembly 110 shows anchor screws respectively anchored to vertebral bone elements 114, 115 and 116. Passing through each anchor screw 111, 112 and 113 is rod 100 having a first end 117 and second opposite free end 118. Free end 117 includes an opening 130 which receives post 98 of arm 88. Working arm 89 is shown engaging rod 100 intermediate screw anchors 111 and 112 via formation 101.

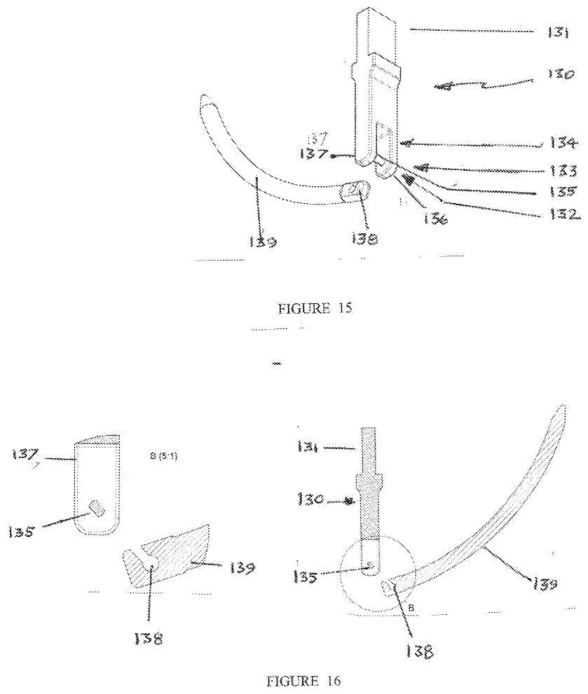

Each of anchor screws locking nuts 120 121 and 122 allow each of screws 111, 112 and 113 to be locked to primary rod 100 or to be free to rotate relative to rod 100. Arm 88 is able to effect a rotation of rod 100 via engagement with formation 98. Arm 89 is able to press down on rod 100 via formation 101. The arrangement in FIG. 15 shows another selection for operation of the arms and rod to effect a desired and/or lordosis of vertebrae of either one of or all of vertebrae 114, 115 and 116.

FIG. 14 shows a side elevation of a rod and screw assembly 110 comprising scissor compressor 80 and connected to its connecting abutments 83 and 84, working arms 89 and 90. Working arm 90 engages rod 100 outside screw anchor 111. Arm 89 engages rod 100 intermediate anchoring screws 112 and 113. In this arrangement, compressor 80 is able to rotate rod 100 in a sagittal plane and/or induce compression. Arm 89 pushes down on rod 100 as arm 90 pulls up on rod 100. This induces translation of vertebra 114 relative to vertebra 115 while compressing those vertebrae. Compressor arm 88 has a circular rod formation 98 that passes through a hole in rod 100. This allows rotation and prevents rod roll over (axial rotation) or rod 100 sliding out of tulips/screws 112, 113 or 114 during compression. Arm 89 which has a two pronged forked formation 101 pushes down on rod Forked formation 101 pushes down on rod 100 while rod formation 98 provides an opposite force enabling compression and at the same time allows lifting of rod 100 enabling translation of vertebra 114.

Lifting the cranial end translates superior vertebrae which unlocks facets. Rod 100 may in another embodiment be lifted by hook formation 97. This translation improves compression even when facets resected. Both Tulips/screw anchors remain unlocked during compression yet the position of rod 100 is still controlled. This is one major advantage of the modified compressor arms according the present invention. Another advantage is that the use of one hand to create the required lordosis allows two rod and screw assemblies to be adjusted simultaneously. Ideally rod 100 has a transverse hole at one end. Rods 50, 51, 52 and 53 have respective openings 54-57 as described in FIG. 6.

According to a method aspect the lordosis is effected according to the following steps: Once rod 100 is engaged with the pedicle screws, one arm 89 with forked formation 101 pushes down on the rod 100 while arm 88 of the compressor engages rod 100 at the cranial end and due to formation 98 resists separation between arm 88 and rod 100. Formation 101 is loaded on rod 100 at a distal end of rod 100. Caps used to lock tulips/screws to rod 100 are added but left loose. Compressor 80 is squeezed and at the same time is lifted at the cranial end and pushed down at the caudal end. This translates superior vertebrae (and top of spine) backwards, unlocking facet joints. This action is enhanced by rod formation 98. Two pedicle screws are compressed iorst and when they have been set and locked, they are then in unison compressed against the third vertebra. Post formation 98 controls the position of rod 100. It presents rollover of rod 100 and applies the compression. Once the screws are locked the compressor is removed and final tightening of the screws takes place. As only one hand is required to rotate and compress and one to lock caps, one person can set the lordosis. A second assistant can set lordosis on the second rod and screw assembly in a similar manner.

FIG. 15 shows an exploded view of an alternative compressor arm 130. Proximal end 131 connects to a compressor (not shown but in a similar manner as previously described,). Distal end 132 includes a formation 133 comprising a two pronged fork 134 and a bridge 135 spanning between forks 136 and 137. Bridge 135 in use engages preformed slot 138 in rod 139. Bridge 135 acts as a cam when interacting with slot 138. Since slot 138 and bridge 135 are disposed at an angle, as arm pulls up rod 139, the change in attitude occasioned by the rotation creates a locking effect between bridge 135 and rod 139 sufficient to resist separation between bridge 135 and rod 139. FIG. 16 shows with corresponding numbering, an enlarged elevation view of distal end 132 of arm 130 and shape of bridge 135 and slot 138 in rod 139.

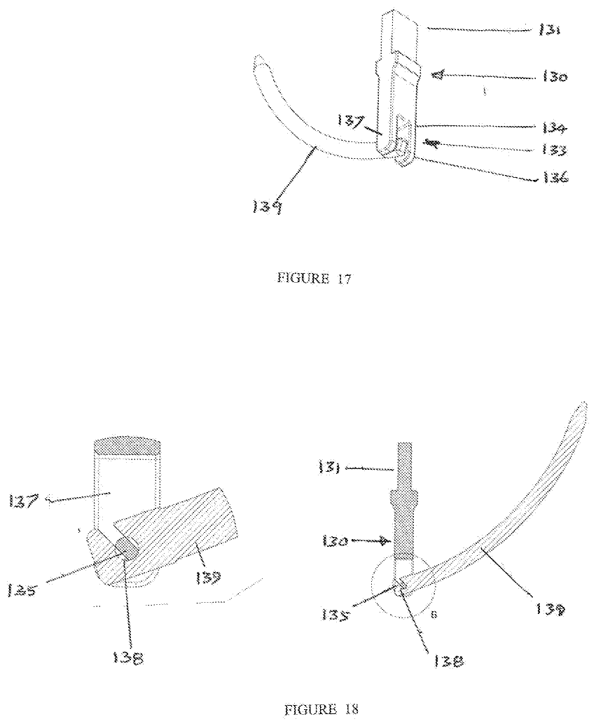

FIG. 17 shows an assembled view of the alternative compressor arm 130. Proximal end 131 connects to a compressor (not shown but in a similar manner as previously described,). Distal end 132 includes a formation 133 comprising a two pronged fork 134 and a bridge 135 (see FIG. 18) spanning between forks 136 and 137. Bridge 135 in use engages preformed slot 138 in rod 139. Bridge 135 acts as a cam when interacting with slot 138. Since slot 138 and bridge 125 are disposed at an angle, as arm pulls up rod 129, the chang4 in attitude occasioned by the rotation creates a locking effect between bridge 135 and rod 139 sufficient to resist separation between bridge 135 and rod 139. FIG. 18 shows an enlarged assembled view of distal end 132 of arm 130 and shape of bridge 135 and slot 138 in rod 139.

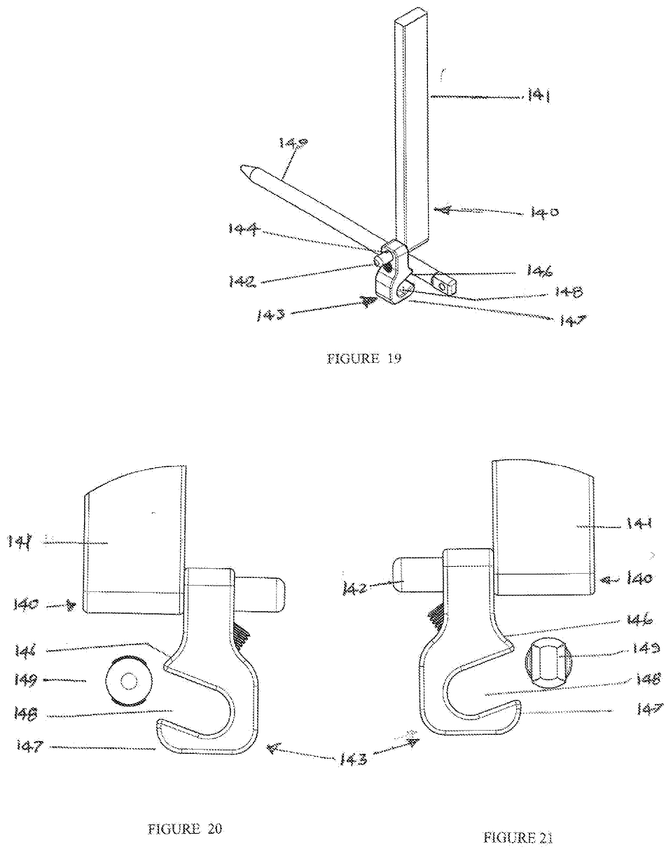

FIG. 19 shows a perspective exploded view of an alternative connection arrangement between a distal end formation of a compressor arm and the primary rod. FIG. 19 shows an abbreviated distal end 140 of compressor arm 141 including a cantilevered post formation 142 depending therefrom. Mounted on post 142 is an attachment 143 which receives post 141 via opening 144. Attachment 143 includes jaws 146 and 147 which define recess 148 which receives and retains therein primary rod 149. Rod 149 is in use mounted on vertebral anchors (not shown) but described earlier. Attachment 143 allows an alternative means to connect arm 141 to rod 149, where the rod 149 does not have any preformed recess or modification to accommodate post formation Attachment 143 allows versatility for a user to attach arm 141 at different positions along rod 149. Post 142 could potentially be placed underneath rod 149 but that is undesirable as the compressor could release and slip off rod 149 potentially causing anatomical damage. Attachment is lockable against rod 149 which ensures that as the compressor is rotated to induce vertebral translation, during compression of vertebrae, arm 141 is securely attached indirectly to rod 149. Post 142 is capable of relative rotation in opening 144 when arm 141 is rotated during use.

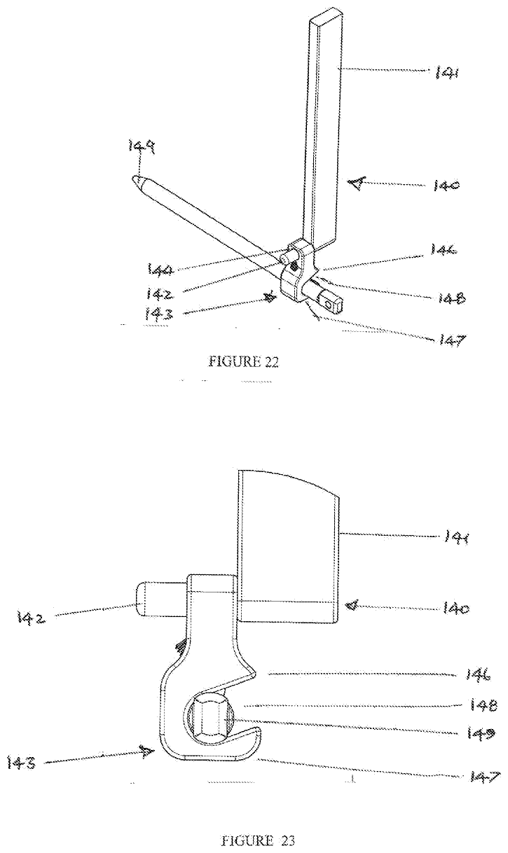

FIG. 20 shows with corresponding numbering an enlarged exploded view of attachment 143 prior to engagement with rod 149. Attachment 143 further comprises a grub screw 150 which when tightened locks rod 149 in recess 148. FIG. 21 shows an opposite side view of the exploded view of FIG. 20. FIG. 22 shows attachment 143 locked to rod 149 and post 142 of compressor arm 141 separated from attachment 143. FIG. 23 shows a perspective exploded view of the alternative connection arrangement between a distal end formation of a compressor arm and the primary rod, with attachment 143 locked into the rod 149. FIG. 23 shows an enlarged end view of the attachment 143 locked to rod 149 and post formation 142 engaged with attachment 143. Grub screw is shown tightened to securely lock attachment 143 to rod 149. Although attachment 143 is described including an opening 144 which receives post 142, it will be appreciated that attachment 143 can be adapted to receive alternative distal end formations on a compressor arm. Also recess 148 can be altered as required to accommodate different rod profiles.

The arrangements described herein allow a surgeon to generate compression, distraction, rotation or a combination of those forces, depending upon a selection from the working arms and selective locking of one or more of the anchors 111, 112 and 113 to rod 100, to manipulate the vertebra into a desired setting.

The system described above with interchangeable working arms allows a surgeon to apply a compressive force between adjacent screws to increase lordosis to improve sagittal balance. The surgeon may distract bone to gain access to the inter vertebral disc spaces for removal of disc, and insertion of spacers and bone graft materials both for support and to aid fusion. The instrumentation described allows one (instead of two) surgeon to apply compression forces and also to tighten the screws in the position determined by the surgeon. The instrumentation according to the present invention (unlike the apparatus of the prior art), also allows the application of a rotational force required to obtain optimal lordosis. Contemplated is a modular rod holder as one of the interchangeable working arms for the compressor. After a rod is inserted a handle is removed and a compressor is attached to a rod holding portion. The holder has the same mechanism to allow the rod to hang loosely via its through hole as if it was connected to a lateral rod.

It will be appreciated by those skilled in the art that numerous variations and modifications may be made to the invention without departing from the overall spirit and scope of the invention broadly described herein.

The claims defining the invention:

1. A compression assembly for positioning spinal vertebra to which are attached anchors on which is mounted a lockable primary rod movable relative to the anchors; the assembly comprising a compression tool capable of inducing a compressive force to said anchors to enable said positioning of said vertebra; the compression tool having first and second arms each having a distal working end which in use, a first of said arms engages the rod; characterised in that each said first and second distal working ends of the tool, each have a formation which co-operates with the rod; the formation on the second arm by co-operation with an anchorage associated with or on the primary rod enabling a concurrent rotation of the rod and translation of a vertebra to which the rod is connected via the vertebral anchors during compression applied to said anchors by the compression tool prior to locking of the rod by the vertebral anchors when vertebrae are in a selected position.

2. A compression assembly according to claim 1 wherein, the formation on the second of said arms which enables the vertebral translation is configured to maintain the engagement with the anchorage on the primary rod during said concurrent compression and translation of the vertebrae.

3. A compression assembly according to claim 2 wherein, the formation on the first arm of the compressor comprises a two pronged fork defining a recess which locates over and receives the primary rod.

4. A compression assembly according to claim 3 wherein, the formation on the first arm is configured to co operate with the rod by accommodating downward pressure on the rod transmitted to the rod via the first arm.

5. A compression assembly according to claim 4 wherein, the formation on the first arm permits relative movement between the formation and the rod.

6. A compression assembly according to claim 5 wherein the first and second arms are detachable from the compression tool.

7. A compression assembly according to claim 6 wherein said rotation of the rod induced by the compressor is along a sagittal plane

8. A compression assembly according to claim 7 wherein, the formation on the second arm of the compressor comprises a two pronged fork with a bridge spanning from one fork to another

9. A compression assembly according to claim 8 wherein, the bridge engages an inclined recess formed in the primary rod.

10. A compression assembly according to claim 7 wherein, the formation on the second arm comprises a circular opening.

11. A compression assembly according to claim 10 wherein, the circular opening on the second arm receives and end of the primary rod thereby retaining the second arm against the rod.

12. A compression assembly according to claim 7 wherein, the formation on the second arm comprises a cantilevered post.

13. A compression assembly according to claim 12 wherein, the cantilevered post is configured to engage an opening in the rod.

14. A compression assembly according to claim 7 wherein, the formation on the second arm comprises a hook.

15. A compression assembly according to claim 14 wherein, the hook engages underneath the rod.

16. A compression assembly according to claim 7 wherein the vertebral anchors are pedicle screws each including a recess which receives and retains the primary rod, the pedicle screws capable of locking the rod once a compression and translation of vertebrae required for lordosis is achieved by the compressor.

17. A compression assembly according to claim 16 wherein, the compression tool has a scissor action enabled by a pivot.

18. A compression assembly according to claim 17 wherein, the rod has a preformed opening.

19. A compression assembly according to claim 18 wherein, the preformed opening is a circular recess at one end of the primary rod.

20. A compression assembly according to claim 19 wherein, the preformed opening is a slot.

21. A compression assembly according to claim 20 wherein, the preformed slot accommodates a bridge in a fork formation in the second arm.

22. A compression assembly according to claim 21 wherein, the preformed slot is configured to prevent separation and withdrawal of the bridge when the rod is under sagittal plane rotation.

23. A compression assembly according to claim 22 wherein, the formation in the primary rod which receives the second arm of the compressor is selected from the following formations: an opening, a groove, a slot, an abbreviation, a recess, a through hole, indent.

24. A compression assembly according to claim 7 wherein the second arm formation cooperates with the rod via a detachable attachment to the rod.

25. A compression assembly according to claim 24 wherein the detachable attachment comprises an attachment body having jaws defining an internal space which receives the primary rod.

26. A compression assembly according to claim 25 wherein, the internal space is generally C shaped

27. A compression assembly according to claim 26 wherein, the jaws are lockable about the primary rod by a locking screw.

28. A compression assembly according to claim 27 wherein, the attachment body further comprises an outstanding formation which receives and retains the formation on the second arm.

29. A compression assembly according to claim 28 wherein, the formation on the second arm comprises a cantilevered post.

30. A detachable arm for detachable attachment to a compression tool used for positioning spinal vertebra to which are attached anchors on which is mounted a lockable primary rod movable relative to the anchors; the compression tool capable of inducing a compressive force to said anchors to enable said positioning of said vertebra; the compression tool having first and second detachable arms, one said detachable arm having a distal working end formation which in use, engages with and cooperates with the primary rod; he formation on the arm by co-operation with an anchorage associated with or on the primary rod enabling a concurrent rotation of the rod and translation of a vertebra to which the rod is connected via the vertebral anchors during compression applied to said anchors by the compression tool prior to locking of the rod by the vertebral anchors when vertebrae are in a selected position.

31. A detachable attachment for detachable attachment to a primary rod to which are attached vertebral anchors on which is mounted the primary rod; a compression tool used for positioning spinal vertebrae capable of inducing a compressive force to said vertebral anchors to enable said positioning of said vertebrae; the compression tool having first and second detachable arms, one said detachable arm having a distal working end formation which in use, engages with and cooperates with the detachable attachment to the primary rod; the formation on the arm by co-operation with the detachable attachment to the primary rod enabling a concurrent rotation of the rod and translation of a vertebra to which the vertebral anchors are during compression applied to said anchors by the compression tool prior to locking of the rod by the vertebral anchors when vertebrae are in a selected position.

32. A compression tool for enabling positioning spinal vertebra by acting on anchors attached to said vertebra and which have mounted thereon a lockable primary rod; the compression tool capable of inducing a compressive force to said anchors to enable said positioning of said vertebra; the compression tool having first and second arms each having a distal working end which in use, engages the rod; characterised in that each said first and second distal working ends of the tool, have a formation which co-operates with the rod; at least one said formations by engagement with the rod resisting separation between the at least one formation and the rod during compression of the screws and rotation of the rod to effect translation of a vertebra during compression applied to said anchors prior to locking of the rod by the anchors.

33. A compression tool according to claim 32 wherein, the formation on the first of said arms comprises a two pronged fork which receives the rod between the forks.

34. A compression tool according to claim 33 wherein, the formation on the second of said arms comprises a cantilevered post which engages an opening in the rod.

35. A compression tool according to claim 33 wherein, the formation on the second of said arms comprises a hole which enables engagement with the rod via and end of the rod.

36. A compression tool according to claim 33 wherein the formation on the second of said arms comprises a hook which engages an underside of the rod.

37. A compression tool according to claim 33 wherein the formation on the second of said arms comprises a two pronged fork having a bridge spanning between the prongs, the bridge engaging a recess in the rod.

38. A compression tool according to claim 37 wherein the recess in the rod is configured to retain the bridge by rotation of the arm relative to the recess.
